SEQUENCE LISTING (15 sequences)

SEQ ID NO: 1 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 2 - protein, 192 aa, Vibrio cholerae
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 3 - protein, 1035 aa, Streptococcus pneumoniae
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 4 - protein, 195 aa, Streptococcus pneumoniae
VIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEK

SEQ ID NO: 5 - protein, 5 aa, Artificial Sequence
  Amino acid linker
ALNGS

SEQ ID NO: 6 - protein, 5 aa, Artificial Sequence
  Amino acid linker
LQALG

SEQ ID NO: 7 - protein, 5 aa, Artificial Sequence
  Amino Acid Linker
GGNSG

SEQ ID NO: 8 - protein, 10 aa, Artificial Sequence
  Amino acid linker
ALNGSGGGSG

SEQ ID NO: 9 - protein, 10 aa, Artificial Sequence
  Amino acid linker
LQALGGGGSL

SEQ ID NO: 10 - protein, 15 aa, Artificial Sequence
  Amino acid linker
ALNGSGGGSGGGGSG

SEQ ID NO: 11 - protein, 438 aa, Pseudomonas aeruginosa
MNTYFDIPHRLVGKALYESYYDHFGQMDILSDGSLYLIYRRATEHVGGSDGRVVFSKLEGGIWSAPTIVAQAGGQDFRDVAGGTMPSGRIVAASTVYETGEVKVYVSDDSGVTWVHKFTLARGGADYNFAHGKSFQVGARYVIPLYAATGVNYELKWLESSDGGETWGEGSTIYSGNTPYNETSYLPVGDGVILAVARVGSGAGGALRQFISLDDGGTWTDQGNVTAQNGDSTDILVAPSLSYIYSEGGTPHVVLLYTNRTTHFCYYRTILLAKAVAGSSGWTERVPVYSAPAASGYTSQVVLGGRRILGNLFRETSSTTSGAYQFEVYLGGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 12 - protein, 106 aa, Pseudomonas aeruginosa
VPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 13 - protein, 489 aa, Artificial Sequence
  Artificial construct
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGALGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 14 - protein, 489 aa, Artificial Sequence
  Artificial construct
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 15 - protein, 489 aa, Artificial Sequence
  Artificial construct
GAMVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

(19) United States
(12) Patent Application Publication
(10) Pub. No.: US 2022/0031816 A1
(43) Pub. Date: Feb. 3, 2022

(54) CANCER TREATMENT

(71) Applicant: OMIDEON LTD, Fife (GB)

(72) Inventors: Helen Connaris, St. Andrews (GB); Graeme Rogers, St. Andrews (GB)

(21) Appl. No.: 17/299,682

(22) PCT Filed: Dec. 6, 2019
(86) PCT No.: PCT/GB2019/053448

§ 371 Date: Jun. 3, 2021

(30) Foreign Application Priority Data
Dec. 6, 2018 (GB) 1819920.8

(51) Int. Cl. A61K 38/47; A61P 35/00

(52) U.S. Cl. CPC A61K 38/47; A61P 35/00

(57) ABSTRACT

The present disclosure provides molecules which modulate cell growth and which have utility in the treatment of certain cancers and as a delivery system for heterologous molecules. The disclosed molecules can also be used to modulate the expression, function and/or activity of certain classes of gene.

### RELATED APPLICATIONS

This application is a 35 U.S.C. § 371 national stage application of PCT Application No. PCT/GB2019/053448, filed Dec. 6, 2019, which claims priority from United Kingdom Patent Application No. 1819920.8, filed Dec. 6, 2018, the contents of each of which are incorporated herein by reference in their entireties. The above-referenced PCT International Application was published in the English language as International Publication No. WO 2020/115495 A1 on Jun. 11, 2020.

### STATEMENT REGARDING ELECTRONIC FILING OF A SEQUENCE LISTING

A Sequence Listing in ASCII text format, submitted under 37 C.F.R. § 1.821, entitled 9013-194_ST25.txt, 38,086 bytes in size, generated on May 20, 2021 and filed via EFS-Web, is provided in lieu of a paper copy. This Sequence Listing is incorporated by reference into the specification for its disclosures.

### FIELD OF THE INVENTION

The present invention provides molecules for use in the treatment of cell proliferation disorders such as cancer, including, for example, glioma cancer, lung cancer, pancreatic cancer and breast cancer.

### BACKGROUND TO THE INVENTION

Some lectins, which are glycoproteins of non-immune origin, exhibit an ability to induce apoptosis in malignant cells and thus demonstrate anti-cancer properties. This phenomenon occurs, in part, through the interaction of these lectins with specific glycan receptors on immune cells (Yau et al., 2015).

One such lectin is Viscumin from mistletoe, which is a toxin that binds to cellular receptors that are glycosylated with α2,6 sialyllactose (Muthing et al., 2002). Viscumin is a 57 kDa heterodimer, comprising of two subunits A and B. The A subunit exerts its toxic effect by disabling ribosomes, thereby interrupting protein production, whereas the B subunit exhibits glycan binding function. The lectin demonstrates picomolar cytotoxicity in vitro and in vivo and has a recommended dose upper limit of 6 μg/kg in clinical trial subjects (half-life of 13 mins) (Zwierzina et al., 2011).

Another plant lectin that has demonstrated an ability to prevent cell migration and growth (and hence an anti-cancer property) is theMaackiaamurensis seed lectin, or MASL (Ochoa-Alvarez et al., 2012; Astarita et al., 2012). This lectin is also cytotoxic exhibiting nanomolar potency (˜300 nM), and exerts its effects through the binding of podoplanin, an α2,3 sialylated mucin-type transmembrane glycoprotein, that is overexpressed in a variety of human cancers (Kato et al., 2005; Schacht et al., 2005; Shibahara et al., 2006).

WO2018/055373 describes the use of sialic acid binding molecules, including carbohydrate binding modules, in methods of modulating cell growth and in the treatment and/or prevention of cell proliferation and/or differentiation disorders.

Due to resistance and a relative lack of current therapeutic options, the treatment of certain specific cancers demands the provision of additional molecules that are well tolerated in their hosts and have therapeutic potential.

### SUMMARY OF THE INVENTION

Molecules which bind sialic acid, including, for example, molecules comprising moieties which belong to the group collectively known as the carbohydrate binding modules (CBM(s) including those belonging to the Family 40 CBM(s) (CBM40(s)), may be used to modulate cell growth and/or development and in the treatment and/or prevention of cell proliferation disorders such as cancer. In particular, the disclosed sialic acid binding molecules (including carbohydrate binding modules CBM(s), especially those belonging to the Family 40 CBM(s) (CBM40(s)) may be used in the treatment and/or prevention of glioma, breast cancer, lung cancer, pancreatic cancer, melanoma (A2058) and ovarian cancer.

The present invention is based on the finding that these molecules and, in particular, those that comprise, consist essentially of or consist of certain Family 40 CBMs, can be used in the treatment of a number of specific cancers. Indeed, data presented herein shows that while molecules comprising CBMs may have a general utility in the treatment of cancer, certain molecules exhibit an unexpected efficacy (or potency) when applied to the treatment and/or prevention of specific cancers. Accordingly, the sialic acid binding molecules (including those comprising CBMs) are particularly useful for the treatment and/or prevention of one or more cancer types, especially those selected from the group consisting of:
(i) glioma;
(ii) breast cancer;
(iii) lung cancer;
(iv) pancreatic cancer;
(v) melanoma; and
(vi) ovarian cancer.

In view of the above, the present disclosure provides sialic acid binding molecules for use in the treatment of one or more cancers selected from the group consisting of:
(i) glioma;
(ii) breast cancer;
(iii) lung cancer;
(iv) pancreatic cancer;
(v) melanoma; and
(vi) ovarian cancer.

Further, the disclosure provides the use of sialic acid binding molecules for the manufacture of a medicament for the treatment of one or more cancers selected from the group consisting of: glioma;
(ii) breast cancer;
(iii) lung cancer;
(iv) pancreatic cancer;
(v) melanoma; and
(vi) ovarian cancer.

The disclosure also provides a method of treating a cancer selected from the group consisting of:
(i) glioma;
(ii) breast cancer;
(iii) lung cancer;
(iv) pancreatic cancer;
(v) melanoma; and
(vi) ovarian cancer.

said method comprising administering a subject in need thereof, a therapeutically effective amount of a sialic acid binding molecule.

The above described sialic acid binding molecules for use, uses, medicaments and/or methods may find application in the treatment and/or prevention of: glioma or breast cancer or lung cancer or pancreatic cancer or melanoma or ovarian cancer. For the avoidance of doubt throughout this specification, the disclosed sialic acid binding molecules include, but are not necessarily limited to sialic acid binding molecules which comprise a CBM40 and/or a sialic acid binding molecule comprising or consisting of or consisting essentially of a VcCBM or a SpCBM

In addition to being useful in the treatment and/or prevention of each of these individual cancers, each of the disclosed sialic acid binding molecules may be useful in the treatment and/or prevention of two, three, four, five or all six of these cancers. For example, the disclosed sialic acid binding molecules may be used in the treatment and/or prevention of one or more cancers selected from the group consisting of glioma; breast cancer; lung cancer; and pancreatic cancer;

Throughout this specification, the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

It should be noted, that as used herein the term “sialic acid binding molecule” embraces molecules and compounds which exhibit an ability to bind to sialic acid. These molecules may take any form and/or belong to any class of molecule or compound (for example, they may be proteins, peptides, carbohydrates, antibodies and the like). Further, the term “sialic acid” embraces all forms of N- or O-substituted neuraminic acid and includes all synthetic, naturally occurring and/or modified forms thereof. Sialic acids may be found as components of cell surface molecules, glycoproteins and glycolipids. Most often, sialic acids are present at the end (terminal regions) of sugar chains connected to cell membranes and/or proteins. For example, some cells of the human upper respiratory tract comprise α-2,6-linked sialic acid receptors and other cells of the upper and lower respiratory tracts comprise α-2,3-linked sialic acid receptors.

The sialic acid family encompasses a number (approximately 50) of derivatives that may result from acetylation, glycolylation, lactonisation and methylation at C4, C5, C7, C8 and C9. All such derivatives are to be embraced by the term “sialic acid”. Furthermore, sialic acids are found linked α(2,3) or α(2,6) to Gal and GalNAc or α(2,8) or α(2,9) to another sialic acid. Accordingly, it is important to understand that while the term “sialic acid” is used throughout this specification, it encompasses all derivatives, analogues or variants (either naturally occurring or synthetically generated) thereof as well as monomers, dimers, trimers, oligomers, polymers or concatamers comprising the same.

Thus, a sialic acid binding molecule of this disclosure (and for use as described above and elsewhere herein) comprises a moiety which exhibits an affinity for sialic acid—including all forms of sialic acid described above and any form of sialic acid present on the surface of a cell, for example a mammalian cell. These various forms of sialic acid may be collectively referred to as “sialic acid moieties”. The sialic acid binding molecules of this disclosure exhibit an affinity for sialic acid and as such they may bind/couple to and/or associate with one or more sialic acid moieties. Thus, the term “sialic acid binding molecule” may further encompass any fragment of a whole sialic acid binding molecule which retains an ability to bind to or otherwise couple or associate with a sialic acid moiety.

Sialic acid binding molecules for use may comprise, consist or consist essentially of, a single sialic acid binding molecule (a monomeric or monovalent molecule, for example) or, alternatively, two or more sialic acid binding molecules—which two or more molecules may be the same or different—a polymeric or multivalent molecule, for example.

A sialic acid binding molecule for use may comprise, consist essentially of or consist of, one or more of the sialic acid binding molecules known as “carbohydrate binding modules” (CBMs). CBMs suitable for use exhibit an affinity for sialic acid. Carbohydrate binding modules are classified into families and CBMs classed as members of the Family 40 CBMs (CBM40) may be useful. The Family 40 CBMs embrace molecules of approximately 200 residues and are often found at the N-terminus of GH33 sialidases. They may also be found inserted in the β-propeller of GH33 sialidases.

Exemplary carbohydrate binding modules for use may comprise the sialic acid binding domain of Vibrio cholerae NanH sialidase (VcCBM: a CBM40) and/or the equivalent (or homologous) domain from Streptococcus pneumoniae NanA sialidase (SpCBM: also a CBM40). Of course, similar or homologous sialic acid binding modules present in other organisms are to be encompassed within the scope of the terms “CBM” and “CBM40”.

An exemplary Vibrio cholerae NanH sialidase amino acid sequence is deposited under accession number A5F7A4 and is reproduced below as SEQ ID NO: 1 (781 amino acids).

MRFKNVKKTA LMLAMFGMAT SSNAALFDYN
ATGDTEFDSP AKQGWMQDNT NNGSGVLTNA
DGMPAWLVQG IGGRAQWTYS LSTNQHAQAS
SFGWRMTTEM KVLSGGMITN YYANGTQRVL
PIISLDSSGN LVVEFEGQTG RTVLATGTAA
TEYHKFELVF LPGSNPSASF YFDGKLIRDN
IQPTASKQNM IVWGNGSSNT DGVAAYRDIK
FEIQGDVIFR GPDRIPSIVA SSVTPGVVTA
FAEKRVGGGD PGALSNTNDI ITRTSRDGGI
TWDTELNLTE QINVSDEFDF SDPRPIYDPS
SNTVLVSYAR WPTDAAQNGD RIKPWMPNGI
FYSVYDVASG NWQAPIDVTD QVKERSFQIA
GWGGSELYRR NTSLNSQQDW QSNAKIRIVD
GAANQIQVAD GSRKYVVTLS IDESGGLVAN
LNGVSAPIIL QSEHAKVHSF HDYELQYSAL
NHTTTLFVDG QQITTWAGEV SQENNIQFGN
ADAQIDGRLH VQKIVLTQQG HNLVEFDAFY
LAQQTPEVEK DLEKLGWTKI KTGNTMSLYG
NASVNPGPGH GITLTRQQNI SGSQNGRLIY
PAIVLDRFFL NVMSIYSDDG GSNWQTGSTL
PIPFRWKSSS ILETLEPSEA DMVELQNGDL
LLTARLDFNQ IVNGVNYSPR QQFLSKDGGI
TWSLLEANNA NVFSNISTGT VDASITRFEQ
SDGSHFLLFT NPQGNPAGTN GRQNLGLWFS
FDEGVTWKGP IQLVNGASAY SDIYQLDSEN
AIVIVETDNS NMRILRMPIT LLKQKLTLSQ
N

The CBM region of SEQ ID NO: 1 is from amino acid residue 25 to 216—this sequence may be SEQ ID NO: 2.

An exemplary Streptococcus pneumoniae NanA sialidase amino acid sequence has been deposited under accession number P62575 and is reproduced below as SEQ ID NO: 3 (1035 amino acids).

MSYFRNRDID IERNSMNRSV QERKCRYSIR
KLSVGAVSMI VGAVVFGTSP VLAQEGASEQ
PLANETQLSG ESSTLTDTEK SQPSSETELS
GNKQEQERKD KQEEKIPRDY YARDLENVET
VIEKEDVETN ASNGQRVDLS SELDKLKKLE
NATVHMEFKP DAKAPAFYNL FSVSSATKKD
EYFTMAVYNN TATLEGRGSD GKQFYNNYND
APLKVKPGQW NSVTFTVEKP TAELPKGRVR
LYVNGVLSRT SLRSGNFIKD MPDVTHVQIG
ATKRANNTVW GSNLQIRNLT VYNRALTPEE
VQKRSQLFKR SDLEKKLPEG AALTEKTDIF
ESGRNGKPNK DGIKSYRIPA LLKTDKGTLI
AGADERRLHS SDWGDIGMVI RRSEDNGKTW
GDRVTITNLR DNPKASDPSI GSPVNIDMVL
VQDPETKRIF SIYDMFPEGK GIFGMSSQKE
EAYKKIDGKT YQILYREGEK GAYTIRENGT
VYTPDGKATD YRVVVDPVKP AYSDKGDLYK
GNQLLGNIYF TTNKTSPFRI AKDSYLWMSY
SDDDGKTWSA PQDITPMVKA DWMKFLGVGP
GTGIVLRNGP HKGRILIPVY TTNNVSHLNG
SQSSRIIYSD DHGKTWHAGE AVNDNRQVDG
QKIHSSTMNN RRAQNTESTV VQLNNGDVKL
FMRGLTGDLQ VATSKDGGVT WEKDIKRYPQ
VKDVYVQMSA IHTMHEGKEY IILSNAGGPK
RENGMVHLAR VEENGELTWL KHNPIQKGEF
AYNSLQELGN GEYGILYEHT EKGQNAYTLS
FRKFNWDFLS KDLISPTEAK VKRTREMGKG
VIGLEFDSEV LVNKAPTLQL ANGKTARFMT
QYDTKTLLFT VDSEDMGQKV TGLAEGAIES
MHNLPVSVAG TKLSNGMNGS EAAVHEVPEY
TGPLGTSGEE PAPTVEKPEY TGPLGTSGEE
PAPTVEKPEY TGPLGTAGEE AAPTVEKPEF
TGGVNGTEPA VHEIAEYKGS DSLVTLTTKE
DYTYKAPLAQ QALPETGNKE SDLLASLGLT
AFFLGLFTLG KKREQ

The CBM region of SEQ ID NO: 3 is from amino acid residue 121 to 305—this sequence may be SEQ ID NO: 4.

Thus, CBMs for use as sialic acid binding molecules in the various aspects and embodiments of this disclosure may comprise a protein or peptide having the sequence of SEQ ID NO: 1 or 2 or a sialic acid binding fragment thereof.

A useful sialic acid binding molecule may comprise a proteinaceous moiety encoded by the sialic acid binding domain of the nanH gene (encoding sialidase) of V. cholerae (as provided by SEQ ID NO: 1) or an equivalent or homologous gene present in another organism (for example the equivalent/homologous nanA sialidase gene of S. pneumoniae: see SEQ ID NO: 3).

A sialic acid binding molecule for use may comprise from about residue 1, 5, 10, 15, 25 or 30 (i.e. from 1-30 or from any amino acid residue there between) to about residue 150, 175, 200, 210, 216, 220-781 (to any residue from 150 to 781 including any residue therebetween) of the V cholerae sialidase molecule of SEQ ID NOS: 1 and 2. For example, a sialic acid binding molecule for use may comprise a peptide having a sequence corresponding to residue 25 to about residue 216 of SEQ ID NO: 1 above.

A further suitable sialic acid binding molecule may comprise a protein or peptide having the sequence of SEQ ID NO: 3 or 4 or a sialic acid binding fragment thereof. For example, a useful sialic acid binding molecule may comprise a proteinaceous moiety encoded by the sialic acid binding domain of the Streptococcus pneumoniae nanA gene (encoding sialidase). For example, a sialic acid binding molecule for use may comprise from about residue 80, 90, 100, 110, 120, 121 to 130 (i.e. from any of about residues 80 to 130 including any residue therebetween) to about residue 250, 275, 300, 305, 310, 320-1035 (i.e. to any residue from about 250-1035 including to about any residue therebetween) of the S. pneumoniae sialidase molecule of SEQ ID NOS: 3 and 4. For example, a sialic acid binding molecule for use may comprise a peptide having a sequence corresponding to residue 121 to about residue 305 of SEQ ID NO: 3 above.

A sialic acid binding molecule for use may comprise one or more CBMs. A suitable sialic acid binding molecule may comprise a single or individual CBM—for example, a single VcCBM or a single SpCBM.

A sialic acid binding molecule for use may comprise a plurality or multiple (i.e. two or more) CBMs. Sialic acid binding molecules which comprise a plurality of CBMs may be termed “multivalent sialic acid binding molecules” or “multivalent CBMs”. A multivalent CBM may, for example, comprise two or more VcCBMs or two or more SpCBMs. A multivalent CBM may comprise a mixture of different CBMs, for example one or more VcCBMs with one or more SpCBMs.

For example, multivalent CBM molecules, (for example a Vc2CBM and/or Vc4CBM molecule) may be prepared as constructs comprising multiple (identical or different) CBMs linked by amino acid/peptide linkers. Each CBM (for example VcCBM and/or SpCBM) may be linked to another (for example VcCBM and/or SpCBM) by, for example, peptides comprising 5, 10 or 15 amino acids. By way of example any one or more of the following peptides may be used to link two or more CBMs to produce a multivalent CBM:

(i) 5 amino acid linkers:
(SEQ ID No: 5)
ALNGS
(SEQ ID No: 6)
LQALG
(SEQ ID No: 7)
GGNSG
(ii) 10 amino acid linkers:
(SEQ ID No: 8)
ALNGSGGGSG
(SEQ ID No: 9)
LQALGGGGSL
(iii) 15 amino acid linkers:
(SEQ ID No: 10)
ALNGSGGGSGGGGSG

An exemplary Vc/Sp 1, 2, 3 or 4 CBM may take the following form:

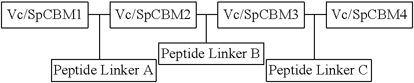

This schematic shall be referred to hereinafter as General Formula 1.

Thus, a Vc/Sp 1, 2 3, 4 CBM molecule may conform to General Formula 1 as set out above, wherein Peptide Linkers A, B and/or C are selected from the linker options presented above as (i), (ii) and/or (iii). For example, a VcCBM or SpCBM may comprise only the CBM module shown as Vc/SpCBM1 shown in formula 1. A Vc2CBM or Sp2CBM may comprise the CBM modules shown as Vc/SpCBM1 and Vc/SpCBM1 shown in formula 1, optionally separated by peptide linker A. A Vc3CBM or Sp3CBM may comprise the CBM modules shown as Vc/SpCBM1, Vc/SpCBM2 and Vc/SpCBM3 shown in formula 1, optionally separated by peptide linkers A and B. A Vc4CBM or Sp4CBM may comprise the CBM modules shown as Vc/SpCBM1, Vc/SpCBM2, Vc/SpCBM3 and Vc/SpCBM4 shown in formula 1, optionally separated by peptide linkers A, B and C.

It should be noted that the term “VcCBM40” or “SpCBM40” embraces not only the complete Family 40 CBM derived from Vibrio cholerae (NanH sialidase) or Streptococcus pneumoniae nanA but also sialic acid binding fragments derived therefrom. Indeed, each of the VcCBM or SpCBM units shown in General Formula 1 may be selected from the group consisting of:

(i) a Vibrio cholerae NanH sialidase CBM;
(ii) a Vibrio cholerae NanH sialidase CBM sialic acid binding fragment thereof.
(iii) a Streptococcus pneumoniae nanA sialidase CBM; and
(iv) a Streptococcus pneumoniae nanA sialidase CBM sialic acid binding fragment thereof

Thus, each of the VcCBM or SpCBM units of the molecule shown in General Formula 1 may be the same or different.

Thus, the various aspects and embodiments of this invention (uses, sialic acid binding molecules for use, methods and medicaments) may exploit sialic acid binding molecules which comprise, consist of or consist essentially of sialic acid binding molecules selected from the group consisting of:
one or more VcCBM(s);
(ii) two (or more) VcCBMs;
(iii) three or four VcCBMs;
(iv) one or more SpCBM(s);
(v) two (or more) SpCBMs;
(vi) a multivalent CBM; and
(vii) a modified CBM.

The sialic acid binding molecules for use may further comprise an oligomerisation domain. Suitable oligomerisation domains may exhibit an ability to self-associate to form multimeric structures, for example trimers. An oligomerisation domain for use may comprise any molecule with the abovementioned oligomerisation properties or any functional fragment thereof. For example, one or more (for example two) sialic acid binding molecules (for example CBMs) may be bound, coupled or fused to an oligomerisation domain—the resulting sialic acid binding molecule::oligomerisation domain “fusion” may then be used (with one or more other such “fusions”) as a molecule for modulating cell growth and/or activity and/or for treating or preventing any of the diseases and/or conditions disclosed herein.

Suitable oligomerisation domains may be derived from, for example, Pseudomonas aeruginosa pseudaminidase. An exemplary Pseudomonas aeruginosa pseudaminidase sequence amino acid sequence has been deposited under accession number PAO579 and is reproduced below as SEQ ID NO: 5 (438 amino acids).

MNTYFDIPHR LVGKALYESY YDHFGQMDIL
SDGSLYLIYR RATEHVGGSD GRVVFSKLEG
GIWSAPTIVA QAGGQDFRDV AGGTMPSGRI
VAASTVYETG EVKVYVSDDS GVTWVHKFTL
ARGGADYNFA HGKSFQVGAR YVIPLYAATG
VNYELKWLES SDGGETWGEG STIYSGNTPY
NETSYLPVGD GVILAVARVG SGAGGALRQF
ISLDDGGTWT DQGNVTAQNG DSTDILVAPS
LSYIYSEGGT PHVVLLYTNR TTHFCYYRTI
LLAKAVAGSS GWTERVPVYS APAASGYTSQ
VVLGGRRILG NLFRETSSTT SGAYQFEVYL
GGVPDFESDW FSVSSNSLYT LSHGLQRSPR
RVVVEFARSS SPSTWNIVMP SYFNDGGHKG
SGAQVEVGSL NIRLGTGAAV WGTGYFGGID
NSATTRFATG YYRVRAWI

The oligomerisation domain of SEQ ID NO: 5 is from amino acid residue 333 to 438—this sequence may be SEQ ID NO: 6.

Thus, an oligomerisation domain for use may comprise from about residue 250, 275, 300, 310, 320, 333, 340 to 350 (i.e. from about residue 250 to about residue 350 including from about any residue therebetween) to about residue 400, 410, 420, 430 or 438 (i.e. to about any residue from about residue 400 residue 438 including to about any residue therebetween) of the P. aeruginosa pseudaminidase trimerisation domain (PaTD) provided by SEQ ID NO: 5. For example, a useful sialic acid binding molecule may exploit an oligomerisation domain comprising residues 333 to 438 of SEQ ID NO: 6.

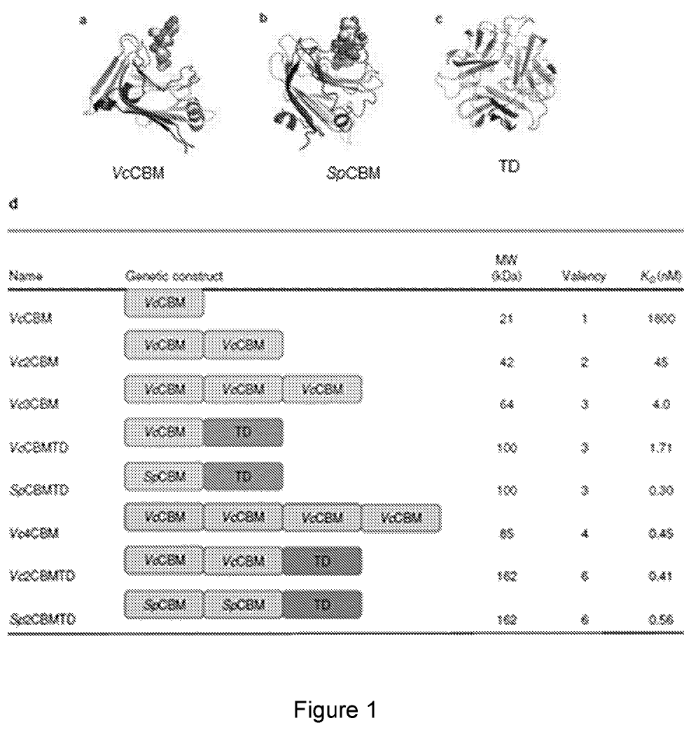

A sialic acid binding molecule for use may comprise one or more of the CBM based molecules presented in FIG. 1. For example, a suitable sialic acid binding molecule may comprise (consist essentially of, or consist of) two or more VcCBMs optionally fused, bound or conjugated to an oligomerisation domain (such as a PaTD or oligomerisation fragment thereof). The sialic acid binding molecule may comprise, consist or consist essentially of two fused (or bound) VcCBMs which are in turn fused to an oligomerisation domain (see, for example, molecule Vc2CBMTD shown in FIG. 1).

Other sialic acid binding domains for use may comprise two or more SpCBMs optionally fused, bound or conjugated to an oligomerisation domain (such as a PaTD or an oligomerisation fragment thereof). Sialic acid binding molecules for use may comprise, consist or consist essentially of two fused (or bound) SpCBMs which are, in turn, fused to an oligomerisation domain (see, for example, molecule Sp2CBMTD shown in FIG. 1).

A sialic acid binding molecule of this disclosure—i.e. sialic acid binding molecules for use in the manufacture of a medicament (for treating cancer and/or the specific cancers described herein), for use in the methods described herein (which methods are for the treatment or prevention of cancer and/or the specific cancers described herein) and compositions for use in the treatment and/or prevention of cancer and/or the specific cancers described herein may comprise, consist essentially of or consist of:
(i) Sp2CBMTD (comprising, consisting essentially of or consisting of: 2 CBMs (CBM40s) from Streptococcus pneumoniae fused to a trimerisation domain;
(ii) Vc2CBMTD (comprising, consisting essentially of or consisting of: 2 CBMs (CBM40s) from Vibrio cholerae fused to a trimerisation domain; and
(iii) Vc4CBM (comprising, consisting essentially of or consisting of: 4 CBMs (CBM40s) from Vibrio cholerae)

Exemplar molecules for use in the various embodiments and aspects described herein are presented as Vc2CBMTD, Vc4CBM and Sp2CBMTD in FIG. 1. Vc2CBM and Vc4CBM may be described as tandem-repeat multivalent proteins based on the Family 40 sialic acid binding domain (CBM) of the nanH gene encoding the sialidase from V cholerae. Sp2CBM may be described as a tandem-repeat multivalent protein based on the family 40 sialic acid binding domain (CBM) of the nanA gene encoding the sialidase from S. pneumoniae.

The terms “sialic acid binding molecule”, “CBM” and/or “CBM40” (all as used herein) may include modified forms of any of the sialic acid binding molecules described herein. Further, the term “modified” embraces molecules which contain one or more mutations relative to a reference sequence.

A “reference sequence” may be any wild type CBM sequence. For example, a reference sequence may comprise, consist essentially of or consist of a wild type family 40 CBM sequence, e.g. the wild type CBM sequences from Vibrio cholerae NanH sialidase or Streptococcus pneumoniae NanA sialidase (it should be appreciated that similar or homologous CBMs (including CBM40s) present in other organisms are to be encompassed within the scope of the term “CBM” and/or as CBM reference sequences). A reference sequence from which a useful sialic acid binding molecule may be derived (including useful multivalent CBMs as described herein) may comprise any of the specific sequences described herein (for example SEQ ID NO: 1, 2, 3, 4 and 5).

For example, a modified CBM sequence for use may be derived from a specific or particular wild type CBM. A useful modified CBM sequence may comprise a wild type CBM sequence which includes one or more mutations.

The one or more mutation(s) may be functional. The mutations may, for example, alter the overall primary sequence of a CBM for use, but may not (substantially) alter the properties of the CBM—thus, while the sequence of a modified CBM may be different from the wild-type sequence from which it is derived, the overall function of the modified CBM is (substantially) identical to that of the wild-type CBM. Alternatively, the one or more mutation(s) may individually (and/or independently) or collectively (for example synergistically) modulate (improve or suppress/inhibit) one or more of the physiological, biological immunological and/or pharmacological properties characteristic of a wild type CBM (for example the wild type CBM from which the modified CBM is derived). In particular, the one or more mutations may:
(i) alter the immunogenicity (or antigenicity) of the CBM; and/or
(ii) alter (for example improve) the efficacy (of the CBM or of any multimeric molecule comprising a modified CBM)′ and/or
(iii) they may modulate (for example improve) the thermostability of the CBM; and/or
(iv) they may modulate (for example improve) the solubility of the CBM; and/or
(v) they may modulate (for example improve) the in vivo half-life of the molecule.

A “mutation” may include any alteration to a wild-type CBM molecule. For example, the term “mutation” may embrace, for example:
(i) one or more amino acid substitution(s) (where one or more of the wild type amino acid(s) is/are swapped or changed for another (different) amino acid—the term “substitutions” would include conservative amino acid substitutions); and/or
(ii) one or more amino acid deletion(s) (where one or more of the wild type amino acid residue(s) are removed); and/or
(iii) one or more amino acid addition(s)/insertion(s) (where additional amino acid residue(s) are added to a wild type (or reference) primary sequence); and/or
(iv) one or more amino acid/sequence inversions (usually where two or more consecutive amino acids in a primary sequence are reversed; and/or
(v) one or more amino acid/sequence duplications (where an amino acid or a part of the primary amino acid sequence (for example a stretch of 5-10 amino acids) is repeated)

Thus, a useful modified CBM (i.e. a CBM for use in the medical uses and methods described herein) may comprise one or more of the mutations described herein.

By way of non-limiting example, the following represent individual units (referred to as “HEX” units) which may be used to make sialic acid binding molecules for the various uses described herein (for example, for use in methods of treating or preventing the various cancers described herein).

### (i) HEX1

### CBM1 (L170T V239A V246G I286A Y292E)-----CBM2 (L170T V239A V246G I286A Y292E)-----TD (S342D L348D R403K)

### (ii) HEX2

### CBM1 (V239A V246G I286A Y292E)-CBM2 (V239A V246G I286A Y292E)-TD (S342D R403K)

(iii) HEX3

CBM1 (V239A V246G I286A)-CBM2 (V239A V246G I286A)-TD (S342D R403K)

### (iv) HEX4

### CBM1 (V239A V246G)-CBM2 (V239A V246G)-TD (S3420

### (v) HEX5

### CBM1 (V239A V246G)-CBM2 (V239A V246G)-TD (R403K)

### (vi) HEX6

### CBM1 (V239A V246G)-CBM2 (V239A V246G)-TD (S342D R403K)

(vii) HEX17

### CBM1 (V239A V246G A162P)-CBM2 (V239A V246G A162P)-TD (S342D R403K)

These sialic acid binding molecules comprising (or consisting essentially of, or consisting of) any of these HEX units may be referred to as hexameric sialic acid binding molecules. A suitable HEX unit may comprise two modified CBMs (denoted CBM1 and CBM2 above) with the specific mutations introduced to each CBM being identified in parenthesis. It should be noted that a “----” symbol indicates an amino acid linker (linking one CBM to another or a CBM to an oligomerisation domain). In each case, the oligomerisation domain (denoted “TD”) present in each HEX unit conjugates the units together as a trimer. While any given hexamer may comprise three identical copies of one of the units described above, one of skill will appreciate that further options are available. For example, a HEX unit may be made up of two CBMs, each having different mutations (the mutations being one or more selected from the options detailed herein).

It will be noted that HEX6 and HEX17 are identical except for the additional A162P mutation. This proline mutation (a substitution for the wild type alanine at residue 162) has been shown to improve thermostability (the single CBM Tm by 3-4° C.). Further information regarding the use of proline mutations may be derived from Fu 2009, ‘Increasing protein stability by improving beta-turns’ (DOI 10.1002/prot.22509) which describes the general approach. The proline mutation does not affect (increase or decrease) the predicted immunogenicity of the CBM molecule, is not located near the other mutations, the N- or C-termini or the ligand binding site. Rather unexpectedly, beyond the modest improvement in thermostability, it was noted that the A162P mutation yields a sialic acid binding molecule exhibiting a marked improvement in in vivo experiments—in particular in comparison to those same experiments conducted using a hexameric molecule comprising other (for example HEX6) HEX units. For example, the modified molecules (in particular, a molecule comprising a HEX17 unit) exhibit modulation over pro-inflammatory cytokines, including, for example, IL-8. Indeed, the modulatory effect (specifically an inhibitory effect) on the production of IL-8 by a molecule comprising a HEX17 unit, was improved over other tested modified molecules.

Relative to the amino acid sequences of Sp2CBMTD (aka “SpOrig”) the amino acid sequence of the HEX6 and HEX17 molecules is:

| | |
|---|---|
| SpOrig | GAMVIEKEDVETNASNGQRVDLSSELDKLKKLENATVHMEFKPDAKAPAFYNLFSVSSAT |
| HEX6 | GAMVIEKEDVETNASNGQRVDLSSELDKLKKLENATVHMEFKPDAKAPAFYNLFSVSSAT |
| HEX17 | GAMVIEKEDVETNASNGQRVDLSSELDKLKKLENATVHMEFKPDPKAPAFYNLFSVSSAT |
| SpOrig | KKDEYFTMAVYNNTATLEGRGSDGKQFYNNYNDAPLKVKPGQWNSVTFTVEKPTAELPKG |
| HEX6 | KKDEYFTMAVYNNTATLEGRGSDGKQFYNNYNDAPLKVKPGQWNSVTFTVEKPTAELPKG |
| HEX17 | KKDEYFTMAVYNNTATLEGRGSDGKQFYNNYNDAPLKVKPGQWNSVTFTVEKPTAELPKG |
| SpOrig | RVRLYVNGVLSRTSLRSGNFIKDMPDVTHVQIGATKRANNTVWGSNLQIRNLTVYNRALT |
| HEX6 | RARLYVNGGLSRTSLRSGNFIKDMPDVTHVQIGATKRANNTVWGSNLQIRNLTVYNRALT |
| HEX17 | RARLYVNGGLSRTSLRSGNFIKDMPDVTHVQIGATKRANNTVWGSNLQIRNLTVYNRALT |
| SpOrig | PEEVQKRSGGGSGVIEKEDVETNASNGQRVDLSSELDKLKKLENATVHMEFKPDAKAPAF |
| HEX6 | PEEVQKRSGGGSGVIEKEDVETNASNGQRVDLSSELDKLKKLENATVHMEFKPDAKAPAF |
| HEX17 | PEEVQKRSGGGSGVIEKEDVETNASNGQRVDLSSELDKLKKLENATVHMEFKPDPKAPAF |
| SpOrig | YNLFSVSSATKKDEYFTMAVYNNTATLEGRGSDGKQFYNNYNDAPLKVKPGQWNSVTFTV |
| HEX6 | YNLFSVSSATKKDEYFTMAVYNNTATLEGRGSDGKQFYNNYNDAPLKVKPGQWNSVTFTV |
| HEX17 | YNLFSVSSATKKDEYFTMAVYNNTATLEGRGSDGKQFYNNYNDAPLKVKPGQWNSVTFTV |
| SpOrig | EKPTAELPKGRVRLYVNGVLSRTSLRSGNFIKDMPDVTHVQIGATKRANNTVWGSNLQIR |
| HEX6 | EKPTAELPKGRARLYVNGGLSRTSLRSGNFIKDMPDVTHVQIGATKRANNTVWGSNLQIR |
| HEX17 | EKPTAELPKGRARLYVNGGLSRTSLRSGNFIKDMPDVTHVQIGATKRANNTVWGSNLQIR |
| SpOrig | NLTVYNRALTPEEVQKRSGGALGVPDFESDWFSVSSNSLYTLSHGLQRSPRRVVVEFARS |
| HEX6 | NLTVYNRALTPEEVQKRSGGSLGVPDFESDWFDVSSNSLYTLSHGLQRSPRRVVVEFARS |
| HEX17 | NLTVYNRALTPEEVQKRSGGSLGVPDFESDWFDVSSNSLYTLSHGLQRSPRRVVVEFARS |
| SpOrig | SSPSTWNIVMPSYFNDGGHKGSGAQVEVGSLNIRLGTGAAVWGTGYFGGIDNSATTRFAT |
| HEX6 | SSPSTWNIVMPSYFNDGGHKGSGAQVEVGSLNIKLGTGAAVWGTGYFGGIDNSATTRFAT |
| HEX17 | SSPSTWNIVMPSYFNDGGHKGSGAQVEVGSLNIKLGTGAAVWGTGYFGGIDNSATTRFAT |
| SpOrig | GYYRVRAWI (SEQ ID NO: 13) |
| HEX6 | GYYRVRAWI (SEQ ID NO: 14) |
| HEX17 | GYYRVRAWI (SEQ ID NO: 15) |

For the avoidance of doubt, the term “HEX17” further embraces the multivalent modified CBM as described above, Vc2CBM comprises, consists essentially of or consists of two Vibrio cholerae NanH sialidase CBM units linked, bound or conjugated together; Vc4CBM comprises, consists essentially of or consists of four Vibrio cholerae NanH sialidase CBM units linked, bound or conjugated together and Sp2CBM comprises, consists essentially of or consists of two Streptococcus pneumoniae NanA sialidase units linked, bound or conjugated together.

The disclosed molecules (for the uses and methods described herein) may be generated using PCR-based cloning techniques and a suitable method for the generation of multivalent molecules of this type is described in, for example, Connaris et al, 2009 (Enhancing the Receptor Affinity of the Sialic Acid-Binding Domain of Vibrio cholerae Sialidase through Multivalency; J. Biol. Chem; Vol. 284(11); pp 7339-7351). For example, multivalent CBM molecules, including the likes of HEX17, Vc2CBM, Vc4CBM and Sp2CBM may be prepared as constructs comprising multiple CBMs linked by amino acid/peptide linkers—such as those described above.

In view of the above, this disclosure provides:

(i) a CBM (including modified CBMs as described above) for use in a method of treating or preventing a cancer selected from the group consisting of: (ii) use of a CBM (including modified CBMs as described above) for the manufacture of a medicament for the treatment and/or prevention of a cancer: and (iii) a method of treating and/or preventing a cancer wherein, for each of (i), (ii) and/or (iii) the cancer is one or more (for example 3, 4, 4, 5 or all six) selected from the groups consisting of:
(i) glioma;
(ii) breast cancer;
(iii) lung cancer;
(iv) pancreatic cancer;
(v) melanoma; and
(vi) ovarian cancer.

Additionally, this disclosure provides:

(1) a CBM (including modified CBMs as described above) for use in a method of treating or preventing a cancer selected from the group consisting of:
(a) glioma;
(b) breast cancer;
(c) lung cancer; and
(d) pancreatic cancer;

(2) use of a CBM (including modified CBMs as described above) for the manufacture of a medicament for the treatment and/or prevention of a cancer selected from the group consisting of:
(a) glioma;
(b) breast cancer;
(c) lung cancer; and
(d) pancreatic cancer;

(3) a method of treating and/or preventing a cancer selected from the groups consisting of:
(a) glioma;
(b) breast cancer;
(c) lung cancer; and
(d) pancreatic cancer.
   said method comprising the step of administering a therapeutically effective amount of a CBM (including modified CBMs as described above) to a subject in need thereof;

(4) a CBM (including modified CBMs as described above) for use in treating and/or preventing a cancer selected from the group consisting of:
(a) glioma;
(b) breast cancer;
(c) lung cancer; and
(d) pancreatic cancer.

In any one of embodiments (i) to (iv) above, the CBM may be (or comprise) a multivalent CBM comprising two or more Family 40 CBMs. In any one of embodiments (i) to (iv) above, the CBM may comprise (consist essentially of, or consist of) two or more VcCBMs and/or SpCBMs optionally fused, bound or conjugated to an oligomerisation domain (such as a PaTD or oligomerisation fragment thereof). The CBM may comprise, consist or consist essentially of two fused (or bound) VcCBMs or two fused (or bound) SpCBMs which are in turn fused to an oligomerisation domain (see, for example, molecule Vc2CBMTD (comprising, consisting essentially of, or consisting of: 2 CBMs (CBM40s) from Vibrio cholerae fused to a trimerisation domain), Vc4CBM (comprising, consisting essentially of or consisting of: 4 CBMs (CBM40s) from Vibrio cholerae and Sp2CBMTD shown in FIG. 1. In any one of embodiments (i) to (iv) above, the CBM may be (or comprise) a modified CBM as described above. It should be noted that CBMs for the various uses, compositions and methods described herein may exploit sialic acid binding molecules which comprise a mix of CBMs, multivalent CBMs and/or modified CBMs as described herein.

In view of the above, this disclosure provides:

(i) the sialic acid binding domain of Vibrio cholerae NanH sialidase (VcCBM) and/or the sialic acid binding domain of Streptococcus pneumoniae NanA sialidase (SpCBM) for use in a method of treating or preventing a cancer selected from the group consisting of:
(a) glioma;
(b) breast cancer;
(c) lung cancer; and
(d) pancreatic cancer.
   said method comprising administering a patient in need thereof a therapeutic amount of the sialic acid binding domain of Vibrio cholerae NanH sialidase (VcCBM) and/or the sialic acid binding domain of Streptococcus pneumoniae NanA sialidase (SpCBM);
   (ii) The sialic acid binding domain of Vibrio cholerae NanH sialidase (VcCBM) and/or the sialic acid binding domain of Streptococcus pneumoniae NanA sialidase (SpCBM) for use in treating and/or preventing a cancer selected from the group consisting of:
(a) glioma;
(b) breast cancer;
(c) lung cancer; and
(d) pancreatic cancer.
   (iii) use of the sialic acid binding domain of Vibrio cholerae NanH sialidase (VcCBM) and/or the sialic acid binding domain of Streptococcus pneumoniae NanA sialidase (SpCBM) for the manufacture of a medicament for the treatment and/or prevention of a cancer selected from the group consisting of:
(a) glioma;
(b) breast cancer;
(c) lung cancer; and
(d) pancreatic cancer.
   (iv) Vc2CBM, Vc4CBM and/or Sp2CBM for use in a method of treating or preventing a cancer selected from the group consisting of:
(a) glioma;
(b) breast cancer;
(c) lung cancer; and
(d) pancreatic cancer.
   said method comprising administering a patient in need thereof a therapeutic amount of the Vc2CBM, Vc4CBM and/or Sp2CBM.
   (v) Vc2CBM, Vc4CBM and/or Sp2CBM for use in treating and/or preventing a cancer selected from the group consisting of:
(a) glioma;
(b) breast cancer;
(c) lung cancer; and
(d) pancreatic cancer.
   (vi) use of Vc2CBM, Vc4CBM and/or Sp2CBM for the manufacture of a medicament for the treatment and/or prevention of a cancer selected from the group consisting of:
(a) glioma;
(b) breast cancer;
(c) lung cancer; and
(d) pancreatic cancer.
(d) pancreatic cancer.
   (vii) Vc2CBM for use in a method of treating or preventing a cancer selected from the group consisting of:
(a) pancreatic cancer; and
(b) breast cancer;
   said method comprising administering a patient in need thereof a therapeutic amount of the Vc2CBM.
   (viii) Vc2CBM for use in treating and/or preventing a cancer selected from the group consisting of:
(a) pancreatic cancer; and
(b) breast cancer;
   (ix) use of Vc2CBM for the manufacture of a medicament for the treatment and/or prevention of a cancer selected from the group consisting of:
(a) pancreatic cancer; and
(b) breast cancer;
   (x) Vc4CBM for use in a method of treating or preventing a cancer selected from the group consisting of:
(a) breast cancer; and
(b) lung cancer.
   said method comprising administering a patient in need thereof a therapeutic amount of the Vc4CBM.
   (xi) Vc4CBM for use in treating and/or preventing a cancer selected from the group consisting of:
(a) breast cancer; and
(b) lung cancer.
   (xii) use of Vc4CBM for the manufacture of a medicament for the treatment and/or prevention of a cancer selected from the group consisting of:
(a) breast cancer; and
(b) lung cancer.
   (xiii) Sp2CBM for use in a method of treating or preventing a cancer selected from the group consisting of:
(a) glioma
(b) breast cancer; and
(c) lung cancer;
   said method comprising administering a patient in need thereof a therapeutic amount of the Sp2CBM.
   (xiv) Sp2CBM for use in treating and/or preventing a cancer selected from the group consisting of:
(a) glioma
(b) breast cancer; and
(c) lung cancer.
   (xv) use of Sp2CBM for the manufacture of a medicament for the treatment and/or prevention of a cancer selected from the group consisting of:
(a) glioma
(b) breast cancer; and
(c) lung cancer.

A subject in need thereof or indeed a subject to be administered a sialic acid binding molecule disclosed herein or a medicament comprising the same, may be any subject suffering (or suspected to be suffering) from a cancer selected from the group consisting of:
(a) glioma;
(b) breast cancer;
(c) lung cancer; and
(d) pancreatic cancer.

The inventors have further noted that Sp2CBMTD and/or Vc2CBMTD may be particularly useful in the treatment and/or prevention of ovarian cancer. As such, the disclosure provides
(i) Sp2CBMTD and/or Vc2CBMTD for use in treating and/or preventing ovarian cancer;
(ii) Use of Sp2CBMTD and Vc2CBMTD in the manufacture of a medicament for the treatment and/or prevention of ovarian cancer; and
(iii) A method of treating or preventing ovarian cancer, said method comprising administering a therapeutically or prophylactically effective amount of a Sp2CBMTD and Vc2CBMTD to a subject in need thereof.

It should be noted, that a subject in need thereof may be a subject suffering from ovarian cancer, diagnosed as having one or more markers, symptoms or predictors of ovarian cancer and/or predisposed or susceptible to, ovarian cancer. The inventors have also noted that Sp2CBMTD has the potential to exhibit a long-lasting and/or residual effect on ovarian cancer cells. That effect (the modulation (for example inhibition) of ovarian cancer cell growth and/or development) may persist and out-last any direct contact between an ovarian cancer cell and the Sp2CBMTD. In other words, the modulatory effect of Sp2CBMTD on ovarian cancer cells may be evident event after the Sp2CBMTD (or drug or medicament comprising the same) has been removed by washing or because of in vivo clearance.

It should be understood that the treatment of one or more of these may involve the use of one or more sialic acid binding molecule(s) of this disclosure to treat, ameliorate or reduce, one or more symptoms of those diseases. By way of example, the symptoms of these specific cancers may include, for example, the presence of tumours and/or cell masses. As such, the sialic acid binding molecules described herein may be used to modulate (for example stop, retard, inhibit or reduce) tumour formation and/or the metastasis thereof. The sialic acid binding molecules may also be used to reduce the overall size of a tumour. Certain tumours, including those that are large and/or aggressive, are often easier to surgically remove if they have first been reduced in size. Typically, chemo- and/or radiotherapy-based treatments might be used to reduce the size of a tumour but treatments such as this may be replaced by and/or supplemented with sialic acid binding molecule-based treatments. In view of the above, the successful treatment of a tumour may therefore be characterised by a reduction in tumour size, a reduction in an observed or detectable/detected level of tumour metastasis, angiogenesis within tumorigenic tissue and/or tissue invasion.

Molecules which exhibit sialic acid molecule binding activity may exhibit an ability to modulate cell (for example a glioma cell, a breast cancer cell, a lung cancer cell and/or a pancreatic cancer cell) growth and/or activity and therefore, without wishing to be bound by theory, the mechanism underpinning the ability of a sialic acid binding molecule (for example, a molecule comprising, consisting essentially of or consisting of one or more CBM(s), CBM40(s), Vc2CBM(s) and/or Sp2CBMs) to affect the size of a (glioma, lung, breast and/or pancreatic) tumour may be rooted in the cell proliferation, differentiation and/or metabolism modulating effects of the molecule.

The term “modulating” may embrace any increase or decrease in one or more aspects of cell growth and/or activity. In other words, a sialic acid binding molecule described herein may either inhibit certain aspects of cell growth and/or activity or may induce or stimulate other aspects of cell growth and/or activity. The terms “growth” and “activity” as applied to cells may embrace processes and/or phenomena associated with one or more of cell proliferation, cell viability, cell migration, cell metabolism, cell differentiation and/or cell morphology/phenotype. The terms “growth” and/or “activity” may further include the response of a cell to certain exogenous and/or endogenous factors or stimuli including, for example, responses to certain compounds of the immune system, cytokines, chemokines and one or more environmental factors (light, temperature, pressure, mechanical stress and the like). Thus, the sialic acid binding molecules disclosed herein may be used to modulate (inhibit, decrease or increase) levels of cell responsiveness.

The inventors have now discovered that the various sialic acid binding molecules described herein act on a number of different cellular pathways and processes. In particular, and without wishing to be bound by theory, it is suggested that sialic acid binding molecules (for example CBM(s), CBM40(s), Vc2CBM(s), Vc4CBM(s) and Sp2CBM(s)) act on various aspects of cellular metabolism, the cell cycle and/or the various mechanisms which control a cell's response to DNA damage. The relevant data is reproduced in Tables 1a and 1b below:

Tables 1a and 1b

(a)
Genes Over-Expressed in
Sp2CBMTG vs. Control Group

| Gene Symbol | Folde Regulation |
|---|---|
| • ERCC5 | 2.35 • |
| ≡ NOL3 | 2.02 ≡ |
| SERPINF1 | 2.88 |
| * SLC2A1 | 4.52 * |
| + SNA13 | 8.88 + |
| >> TEP1 | 2.86 >> |
| > VEGFC | 4.85 > |

(b)
Genes Under-Expressed in
Sp2CBMTD vs. Control Group

| Gene Symbol | Fold Regulation | |
|---|---|---|
| * ARNT | | −2.32 * |
| * ATPSA1 | | −4.72 * |
| ○ AURXA | | [symbol] +501 |
| ○ [symbol] | | −4.33 ○ |
| ≡ CASP2 | | −4.88 ≡ |
| ≡ [symbol] | | [symbol] ≡ |
| ○ CCNO3 | | −8.91 ○ |
| ○ [symbol] | | [symbol] ○ |
| ∧ [symbol] | | −14.67 ∧ |
| • COB2 | | [symbol] • |
| • [symbol] | [symbol] | [symbol] >> |
| ○ E2F4 | | −8.52 ○ |
| • ERCC3 | | −7.66 • |
| * [symbol] | | [symbol] * |
| [symbol] | | −3.52 |
| * LOHA | | −2.58 * |
| ○ MCM2 | | [symbol] ○ |
| ○ [symbol] | | [symbol] ○ |
| ○ [symbol] | | −3.47 ○ |
| • [symbol] | | [symbol] • |
| ○ [symbol] | | −4.49 ○ |
| [symbol] | | −2.88 |
| < [symbol] | | −13.13 < |
| * [symbol] | | [symbol] * |
| ○ [symbol] | | [symbol] ○ |
| << ACT3 | | [symbol] << |
| * GAPDH | | −2.23 * |
| * [symbol] | | −2.58 * |

*: cell metabolism
○: cell cycle [symbol]
: tumour marker
•: DNA Damage response
+: transcriptional rpressor
≡: apoptosis
∧: cell adhesion
<<: motility
<: microtubule regulation
>: growth factor [symbol]
indicates data missing or illegible when filed

Tables 1a and 1b show the results of an analysis of RNA gene expression in CBM treated lung cancer cells. The results show that in these (CBM treated) cells, 7 genes were found to be overexpressed (see Table 1a) and 30 were found to be under-expressed (see Table 1b). A further analysis shows that these genes represent genes involved in cell cycle, metabolism and DNA repair pathways. The results further show that genes involved in the DNA damage response (marked with “•”), the cell cycle (marked with “∘”) and cellular metabolism (marked with “*”) appear to be most affected by the CBM.

Accordingly, the various sialic acid binding molecules described herein, and in particular the CBMs, CBM40s, VcCBM(s), SpCBM(s), Vc2CBM(s), Vc4CBM(s) and/or Sp2CBM(s) may be used in the treatment and/or prevention of cancers associated with aberrant, defective, deficient or abnormal cell metabolism, cell cycle function(s) and/or DNA damage repair processes. An aberrant defective, deficient or abnormal cell metabolic process or function may exhibit a reduced level of activity or function as compared to the level of activity or function exhibited by the same cell metabolic process or function in a normal cell of the same type. A “normal” cell may be a healthy, non-cancerous cell. The cell may be a healthy lung cell, a glial cell, an astrocyte, a pancreas cell or a mammary cell.

The effect of the disclosed sialic acid binding molecules (including the molecules which comprise the various CBMs and CBM40s described herein) on gene expression may be exploited in in vitro and in vivo methods of modulating the expression, function and/or activity of one or more genes. The described in vitro and in vivo methods may comprise a step in which a cell or cells is contacted with a sialic acid bind molecule (such as any of those described herein) under conditions which permit the sialic acid binding molecule to bind to a sialic acid containing receptor on the cell surface. The term modulated (as used in relation to these in vitro and in vivo methods) may refer to any increase or decrease in any aspect of the activity, function and/or expression of a gene. For example, a gene may be modulated such that the level of expression of the gene may be increased described relative to the level of expression of the same gene in a cell which has not been contacted with a sialic acid binding molecule as described herein.

In view of the above, the described in vitro and in vivo methods may be used to modulate the expression, function and/or activity of one or more genes related to DNA damage repair. For example, the sialic acid binding molecules described herein may be used to modulate (for example increase or decrease) the expression of one or more of the ERCC5, DDB2, DKC1, ERCC3 and/or POLB genes. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to:

(a) increase the expression of the ERCC5 gene; and/or
(b) decrease the expression of one, more or all of the gene(s) selected from the group consisting of:
(1) DDB2;
(2) DKC1;
(3) ERCC3; and
(4) POLB.

The described in vitro and in vivo methods may be used to modulate the expression, function and/or activity of one or more genes related to cell metabolism. For example, the sialic acid binding molecules described herein may be used to modulate (for example increase or decrease) the expression of one or more of the SLC2A1, ARNT, ATP5A1, COX5A, G6PD, LDHA, UQCRFS1, GAPDH and/or HPRT1 genes. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to:

(a) increase the expression of the SLC2A1 gene; and/or
(b) decrease the expression of one, more or all of the gene(s) selected from the group consisting of:
(1) ARNT;
(2) ATP5A1;
(3) COX5A;
(4) G6PD;
(5) LDHA;
(6) UQCRFS1;
(7) GAPDH; and/or
(8) HPRT1.

The described in vitro and in vivo methods may be used to modulate the expression, function and/or activity of one or more genes related to the cell cycle. For example, the sialic acid binding molecules described herein may be used to modulate (for example increase or decrease) the expression of one or more of the AURKA, BMP1, CCND3, CDC20, E2F4, MCM2, MK167, PINX1, SKP2 and/or WEE1 genes. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to decrease the expression of one, more or all of the gene(s) selected from the group consisting of:
(1) AURKA,
(2) BMP1;
(3) CCND3;
(4) CDC20;
(5) E2F4;
(6) MCM2;
(7) MK167;
(8) PINX1;
(9) SKP2; and/or
(10) WEE1.

The described in vitro and in vivo methods may be used to modulate the expression, function and/or activity of one or more genes related to (or encoding) transcriptional repressor(s). For example, the sialic acid binding molecules described herein may be used to modulate (for example increase or decrease) the expression of the SNAI3 gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to increase the expression of the SNAI3 gene.

The described in vitro and in vivo methods may be used to modulate the expression, function and/or activity of one or more genes related to apoptosis. For example, the sialic acid binding molecules described herein may be used to modulate (for example increase or decrease) the expression of one or more of the ANOL3, CASP2 and/or CASP7 genes. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to:

(a) increase the expression the NOL3 gene; or
(b) decrease the expression of one, more or all of the gene(s) selected from the group consisting of:
(1) CASP2; and/or
(2) CASP7;

The described in vitro and in vivo methods may be used to modulate the expression, function and/or activity of one or more genes related to cell adhesion. For example, the sialic acid binding molecules described herein may be used to modulate (for example increase or decrease) the expression of the CDH2 gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to decrease the expression of the CDH2 gene.

The described in vitro and in vivo methods may be used to modulate the expression, function and/or activity of one or more genes related to telomerase activity. For example, the sialic acid binding molecules described herein may be used to modulate (for example increase or decrease) the expression of the TEP1 gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to increase the expression of the TEP1 gene.

The described in vitro and in vivo methods may be used to modulate the expression, function and/or activity of one or more genes related to cell motility. For example, the sialic acid binding molecules described herein may be used to modulate (for example increase or decrease) the expression of the ACTB gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to decrease the expression of the ACTB gene.

The described in vitro and in vivo methods may be used to modulate the expression, function and/or activity of one or more genes related to microtubule regulation. For example, the sialic acid binding molecules described herein may be used to modulate (for example increase or decrease) the expression of the STMN1 gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to decrease the expression of the STMN1 gene.

The described in vitro and in vivo methods may be used to modulate the expression, function and/or activity of one or more genes related to (or encoding) cellular growth factors. For example, the sialic acid binding molecules described herein may be used to modulate (for example increase or decrease) the expression of the VEGFC gene. For example, a sialic acid binding molecule or a molecule comprising (i) one or more CBMs; (ii) one or more CBM40s; (iii) one or more VcCBM(s); (iv) one or more SpCBMs; (v) Vc2CBM and/or (vi) Sp2CBM may be used to increase the expression of the VEGFC gene.

Molecules which bind sialic acid and in particular sialic acid which is part of a cell bound or cell surface receptor, may find further application as moieties which may be conjugated, bound or joined to or associated with, other some heterologous molecule for the purpose of targeting or delivering that heterologous molecule to some tissue or cell. This utility is based on the finding that beyond simply binding to the sialic acid component which is present within certain cell surface receptors, the disclosed sialic acid binding molecules can become internalised (within the cell) after binding.

This finding leads directly to the suggestion that a sialic acid binding molecule of this disclosure can be used to deliver an entity which is bound, conjugated, associated or joined thereto, not just to the cell surface, but also to the inside of a cell. In other words, any of the disclosed sialic acid binding molecules (in particular those comprising, consisting essentially of or consisting of CBMs, CBM40s, Sp2CBMTD and Vc2CBMTD) may be exploited as conjugated/delivery vehicles to deliver a payload into a cell, for example a cancer cell.

A sialic acid::heterologous entity complex (in which the heterologous entity is somehow bound, joined, conjugated to, or associated with, a sialic acid binding molecule) may otherwise be known as a “therapeutic warhead” or “conjugate” and for convenience, we shall refer hereinafter to all such complexes as “conjugate(s)”.

Without wishing to be bound by theory, the presence of sialic acid in certain cell receptors allows conjugates of the type described herein, to be exploited as a means to deliver a heterologous molecule (conjugated to a sialic acid binding molecule) to at least the surface of a cell expressing a sialic acid containing receptor. However, it is now known that after binding (between the conjugate and the sialic acid containing receptor) the conjugate can become internalised; in effect this delivers the heterologous moiety (for example a cytotoxic or cytostatic moiety) to the inside of a cell.

The conjugates described herein (that is, conjugated sialic acid binding molecules) may be useful in the treatment of cancer, where sialic acid binding molecules (with affinity for sialic acids expressed on cancerous cells and/or tumours) may be used to direct therapeutic (for example cytostatic and/or cytotoxic moieties) not only to the surface of specific cells, but also inside those cells. One of skill will appreciate that certain therapeutic agents (for example cytostatic and/or cytotoxic agents) are most effective if they can be internalised. Many (if not all) of these therapeutic agents cannot themselves affect their own internalisation—as such, conjugation to molecules like the disclosed sialic acid binding molecules represents one way of achieving the necessary internalisation.

By way of example, a sialic acid binding molecule as described herein (including any of the CBM based molecules) may be conjugated to one or more agents which are, for example, therapeutic, cytostatic and/or cytotoxic.

For convenience, the conjugated agent shall be generically referred to as a “heterologous agent” (the term “heterologous” meaning something which is not the sialic acid binding molecule). The heterologous agent may comprise, for example, a therapeutic, cytostatic and/or cytotoxic moiety which may be conjugated to some part of the sialic acid binding molecule. For example, the heterologous agent may be conjugated to one or both ends of the sialic acid binding molecule. The heterologous moiety may be additionally or alternately conjugated (or even fused) to an internal portion of the sialic acid binding molecule. It will be appreciated that however the heterologous agent is to be conjugated to the sialic acid binding molecule, the agent (nor its conjugation) should not (substantially) interfere with, ablate or reduce the sialic acid binding property of the sialic acid binding molecule.

As stated, the heterologous agent may be a drug useful in the treatment of a disease which affects a cell or tissue comprising sialic acid. For example, such a cell or tissue may express a receptor which contains sialic acid. The to be conjugated to a sialic acid binding molecule of this invention, may be a chemotherapeutic drug for use in the treatment of cancer and the like. The heterologous agent may be a cytotoxic moiety capable of killing or inducing apoptosis in, a cell. The heterologous agent may be a cytostatic agent capable of preventing a cell from growing, differentiating or dividing. A cytostatic agent may arrest the cell cycle. The heterologous agent may comprise a molecule which is able to recruit specific cells to or into a particular tissue. For example, the heterologous agent may be, for example, a T cell receptor (TCR) which may be used as a means to recruit T cells to, for example a tumour or cancerous tissue.

Thus, the disclosure relates to sialic acid binding molecule conjugates. In one embodiment the disclosure provides sialic acid binding molecules which are conjugated (bound or joined to, or associated with) one or more agents selected from the group consisting of:
a therapeutic agent;
a cytotoxic agent; and
a cytostatic agent.

The sialic acid binding component of any of the conjugates described herein may be (or may comprise) a carbohydrate binding module (a CBM). The sialic acid binding component of any of the conjugates described herein may comprise a CBM which is a member of the family 40 CBMs (a CBM40). The sialic acid binding component of any of the conjugates described herein may comprise one or more VcCBM molecules. Where the sialic acid binding molecule of the conjugate comprises one or more VcCBM molecules, the sialic acid binding molecule may optionally further comprise an oligomerisation domain, e.g. a trimerisation domain (as described herein). The sialic acid binding component of any of the conjugates described herein may comprise, consist of or consist essentially of Vc2CBM, Vc4CBM, or Vc2CBMTD.

The sialic acid binding component of any of the conjugates described herein may comprise one or more SpCBM molecules. Where the sialic acid binding molecule of the conjugate comprises one or more SpCBM molecules, the sialic acid binding molecule may optionally further comprise an oligomerisation domain, e.g. a trimerisation domain (as described herein). The sialic acid binding component of any of the conjugates described herein may comprise, consist of or consist essentially of Sp2CBM or Sp2CBMTD.

The present disclosure may provide compositions for use in the various uses, medicaments and methods described herein. As such, any of the sialic acid binding molecule(s) described herein may be formulated for use. The sialic acid binding molecules (including those comprising, consisting essentially of or consisting of one or more CBM(s), CBM40(s), Vc2CBM(s), Vc4CBM(s) and/or Sp2CBM(s)) may also be formulated for use, for example for use in the treatment and/or prevention one or more cancers selected from the group consisting of:
(a) glioma;
(b) breast cancer;
(c) lung cancer; and
(d) pancreatic cancer.

For convenience, and with reference to the section below describing compositions, formulations and the like it should be noted that both sialic acid binding molecules as described herein and any conjugates comprising the same (for example sialic acid binding molecule::drug conjugates/fusions) shall be included under the general term “sialic acid binding molecule”.

A sialic acid binding molecule (or molecules) may be formulated for use and as a therapeutic or pharmaceutical composition. The various compositions may comprise one or more of the sialic acid binding molecules described herein and any given treatment may require the administration (together, concurrently or separately) of one or more of these compositions.

The various sialic acid binding molecules described herein may be formulated for enteral (including oral), parenteral and/or topical administration and one of skill will appreciate that the precise formulation may vary depending on the route of administration.

Pharmaceutical compositions according to the present invention may be prepared conventionally, comprising substances that are customarily used in pharmaceuticals and as described in, for example, Remington's The Sciences and Practice of Pharmacy, 22nd Edition (Pharmaceutical Press 2012) and/or Handbook of Pharmaceutical Excipients, 7th edition (compiled by Rowe et al, Pharmaceutical Press, 2012)—the entire content of all of these documents and references being incorporated by reference.

A therapeutic or pharmaceutical composition of this disclosure (that is a composition comprising a sialic acid binding molecule and for use in any of the medicaments or methods described herein—including the methods of or medicaments for, modulating cell growth and/or activity and/or treating cancer) may be formulated together with one or more pharmaceutically acceptable excipients, carriers, adjuvants and buffers. The compositions can be administered, e.g. orally (including mucosally), parentally, enterally, intramuscularly, subcutaneously, intravenously or via any other routes useful to achieve the desired effect (in this case effects which include, modulation of cell growth/activity, treatment or prevention of diseases/conditions associated with the same and/or cancer and/or modulation of tumour growth). As stated, depending on the chosen route of administration, the exact composition of the formulation may vary.

A therapeutic or pharmaceutical formulation comprising a sialic acid binding molecule and for administration to a subject may be coated, encapsulated or enveloped in a material which protects the sialic acid binding molecule from the action of enzymes, acids and other natural compounds/conditions (including, for example, compounds (including antibodies), cells and processes of the immune system) which may inactivate or denature the compound and/or its sialic acid binding properties.

Among the various standard and conventional excipients that may be available for use in compositions comprising sialic acid binding molecules, are those pharmaceutically acceptable organic or inorganic carrier substances which are suitable for parenteral, enteral, oral (including mucosal) and other routes of administration that do not deleteriously react with the sialic acid binding molecule(s).

Where the sialic acid binding molecules are to be formulated for parental administration, the compositions may be sterile.

The composition may comprise an oil-based or aqueous solution, a suspension and/or an emulsion.

In other embodiments, the composition may take the form of an implant, such as for example a (dissolvable or biodegradable) film, pessary or implant (including suppositories).

The pharmaceutical preparations comprising sialic acid binding molecules may be mixed with stabilizers, wetting agents, emulsifiers, salts (for use in influencing osmotic pressure), buffers and/or other substances that do not react deleteriously with the active compounds.

One or more of the sialic acid binding molecules described herein may be formulated for and administered, orally. As stated, oral administration would include mucosal administration which would itself would include administration intranasally and/or by inhalation.

Compositions for use may include solid dosage forms which are suitable for oral administration. These may include, for example capsules, tablets, pills, powders, and granules. In any given solid dosage form, the sialic acid binding molecule (or any conjugate comprising the same) may be admixed with at least one inert pharmaceutically-acceptable excipient.

Examples of suitable excipients will be known to one of skill in this field but may include, for example fillers or extenders, humectants, wetting agents, binders, disintegrating agents, solution retarders, absorption accelerators, adsorbents, lubricants or mixtures thereof. A tablet, pill or capsule may further comprise a buffering agent. Solid dosage forms such as tablets, dragees, capsules, pills and/or granules also can be prepared with coatings and shells, such as coatings which protect against the gastrointestinal environment and/or stomach acid.

A solid dosage form may contain opacifying agents and can also be formulated so as to ensure the delayed release of the active agent (in this case the sialic acid binding molecule or a conjugate comprising the same) in or to a specific part of the intestinal tract.

Solid compositions for oral administration can be formulated in a unit dosage form, each dosage containing an appropriate dose of the sialic acid binding molecule (or conjugate comprising the same). The exact amount of sialic acid binding molecule (or conjugate comprising the same) contained within any given solid dosage form will vary depending on the intended use. a solid composition may contain a “unit dose”—a unit dose containing a quantity of sialic acid binding molecule (or conjugate containing the same) calculated to produce the desired effect (for example modulation of cell growth and/or activity) over the course of a treatment period.

Liquid dosage forms for oral administration may (as stated) include emulsions, solutions, suspensions, syrups, and elixirs. In addition to the compound or composition, the liquid dosage forms may contain inert diluents commonly used in the art, such as water or other solvents, solubilizing agents and emulsifiers.

The sialic acid binding molecules may be used in any suitable amount. As stated, the sialic acid binding molecules may be formulated for oral, mucosal or parenteral administration and as such, the precise formulation may depend on the intended route of administration. The amount of sialic acid binding molecule present in any given dose may be in the region of 0.1 μg-1000 μg. For example, amounts of about 0.1 μg, 0.2 μg, 0.3 μg, 0.4 μg, 0.5 μg, 1 μg, 10 μg, 20 μg, 25 μg, 50 μg, 100 μg, 200 μg, 300 μg, 400 μg, 500 μg, 600 μg, 700 μg, 800 μg or about 900 μg. Higher amounts of any of the sialic acid binding molecules may also be used including amounts from about 1 mg to about 1000 mg. For example (and depending on the route of administration and/or the organism to which the sialic acid binding molecule is to be administered (for example a human)) about 1 mg, 2 mg, 3 mg, 4 mg, 5 mg, 10 mg, 20 mg, 25 mg, 50 mg, 100 mg, 200 mg, 300 mg, 400 mg, 500 mg, 600 mg, 700 mg, 800 mg or about 900 mg of a sialic acid binding molecule may be administered. The selected amount of sialic acid binding molecule may be formulated in a specific volume of a pharmaceutically acceptable excipient, diluent and/or buffer. The actual volume required may depend on the dose of sialic acid molecule to be administered, the route of administration and/or the duration of any treatment regime. The volume of excipient, diluent or buffer may be about 10 μl to 5 ml. For example, the required amount of sialic acid binding molecule (CBM) may be combined (or formulated) with about 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 200 μl, 250 μl, 300 μl, 400 μl, 500 μl, 600 μl, 700 μl, 800 μl, 900 μl, 1 ml, 2 ml, 3 ml or 4 ml. For example, an amount of 100 μg sialic acid binding molecule may be combined with about 250 μl of excipient to yield a final concentration of sialic acid concentration of 400 μg/ml. Doses at concentrations of about 0.1 μg/ml-1 mg/ml may be used including, for example, doses at 5 μg/ml, 10 μg/ml, 20 μg/ml, 25 μg/ml, 50 μg/ml, 100 μg/ml, 200 μg/ml, 300 μg/ml, 500 μg/ml, 600 μg/ml, 700 μg/ml, 800 μg/ml or 900 μg/ml.

In use, a dose of a sialic acid binding molecule, administered as part of the treatment and/or prevention of a cell proliferation and/or differentiation disorder (for example cancer), may be administered multiple times over a number of days, weeks months or years. For example, after an initial (or first) administration, a dose of a sialic acid binding molecule may be administered again at about (+/−1 or 2 days) 3, 4, 5, 6, 7, 17, 21, 28 and/or 35 days later. On any given day, a specific dose of a sialic acid binding molecule may be administered 1, 2, 3 or more times. Each time, the sialic acid binding molecule may be administered (by whatever route is considered best to affect a suitable treatment or to induce prophylaxis against the development of a cell proliferation and/or differentiation disorder.

### DETAILED DESCRIPTION

The present invention will now be described in detail by reference to the following Figures which show:

FIG. 1: Building blocks of the multivalent CBM forms and their affinities for sialic acid. a, VcCBM, residues 25-216 of the V cholerae sialidase (PDB:1w0p) with α-2,3-sialyllactose drawn as spheres. b, SpCBM, residues 121-305 of S. pneumoniae NanA sialidase with α-2,3-sialyllactose (PDB:4clw). c, TD, the trimerisation domain, residues 333-438, of the P. aeruginosa pseudaminidase (PDB:2w38) in rainbow colours; the other two monomers in single colours. d, Multivalent forms: their molecular weights, valencies and binding affinities for α2,3-sialyllactose as determined by surface plasmon resonance (SPR) at 25° C. (K_{D} values for VcCBM, Vc2CBM and Vc3CBM had been reported previously (Connaris et al, 2009)). Tandem repeat CBMs, and oligomeric CBMs fused to TD are linked by a 5-amino linker (details in Connaris et al, 2014).

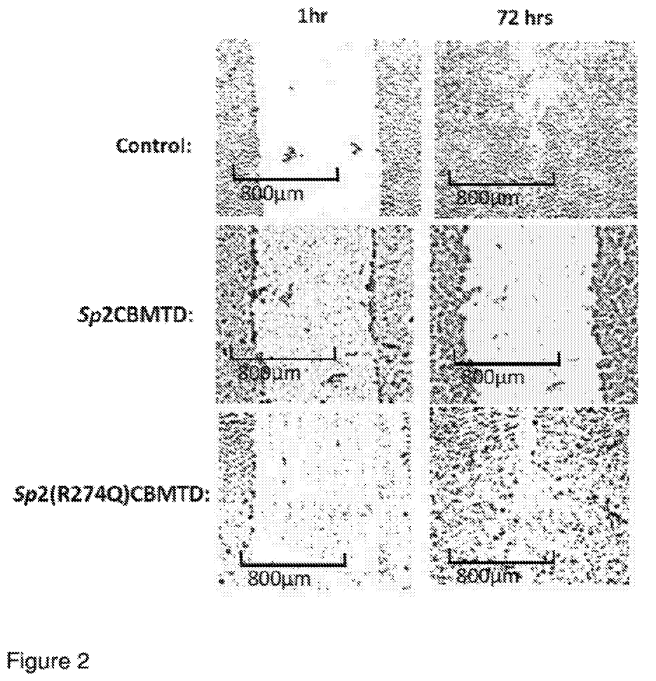

FIG. 2: Wound healing assay. Scratch assay data for human lung carcinoma cell line (A549) after treatment with either PBS, Sp2CBMTD or Sp2(R274Q)CBMTD; a null sialic acid binding mutant (100 μg (400 μg/ml). Images represent cell wound closure after 72 hours.

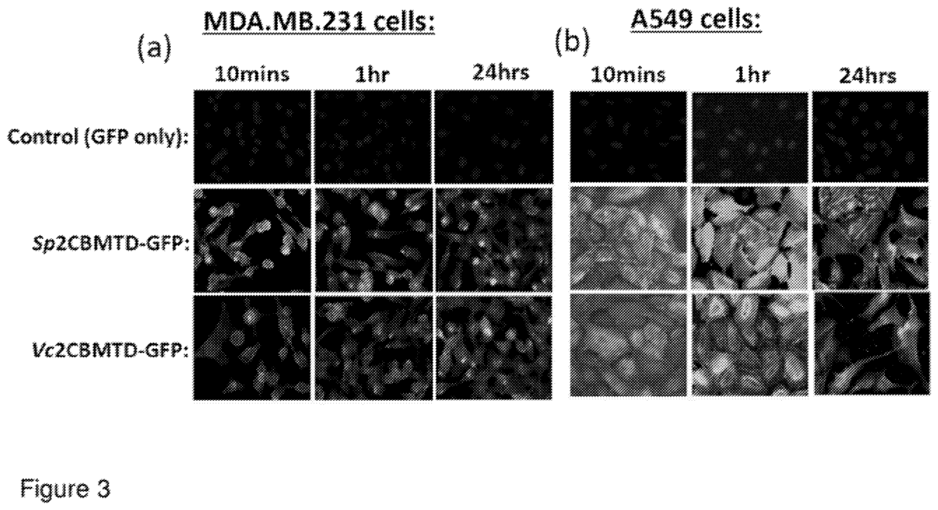

FIG. 3: Visualising the binding of mCBM40 on cancer cells using GFP-tagged variants. (a). MDA.MB.231 cell line incubated with GFP (1 mg/mL), Sp2CBMTD-GFP (25 μg/ml) and Vc2CBMTD-GFP (25 μg/ml) for 10 minutes, 1 hour and 24 hours. Cells then washed with PBS (2×500μ1), fixed using formalin and stained using DAPI. (b) A549 cell line incubated with GFP (1 mg/ml), Sp2CBMTD-GFP (25 μg/ml) and Vc2CBMTD-GFP (25 μg/ml) for 10 minutes, 1 hour and 24 hours. Cells then washed with PBS (2×500 μl), fixed using formalin and stained using DAPI. Cells were then imaged using an EVOS microscope. This Figure shows that Sp2CBMTD-GFP and Vc2CBMTD-GFP can be exploited as conjugated/delivery vehicles for the delivery of a heterologous molecule (in this example GFP) into a cell, for example a cancer cell.

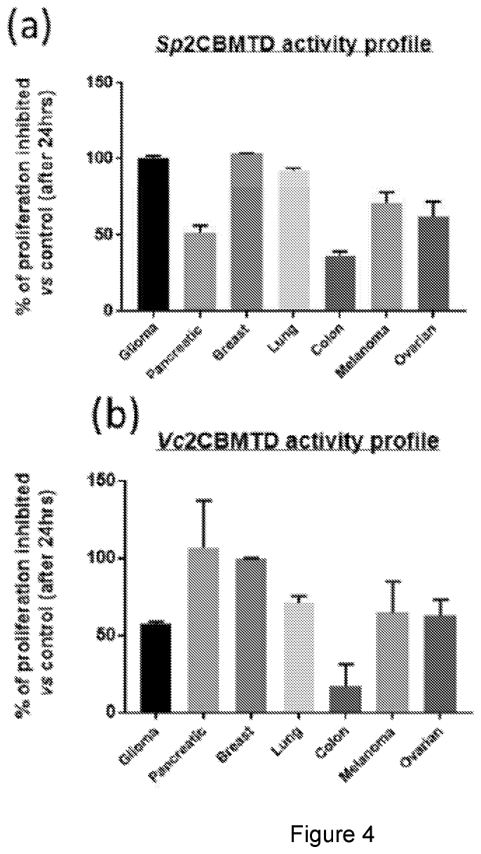

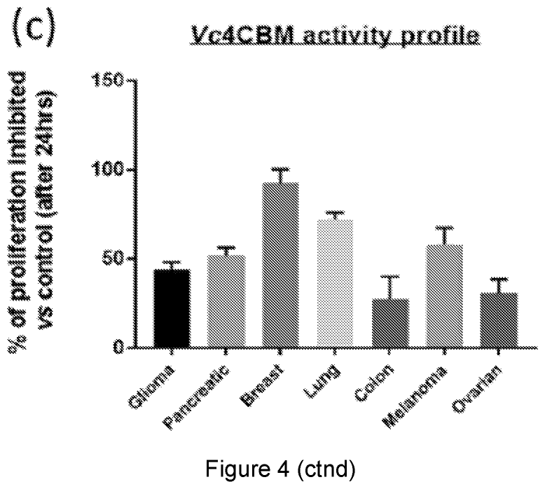

FIG. 4: proliferation data (BrdU incorporation assay). Different cancer cell lines (Glioma (T98G), Pancreatic (PANC1), Breast (MDA.MB.231), Lung (A549), Colon (SW620), Melanoma (A2058) and Ovarian (PE014)) were treated with Sp2CBMTD (400 μg/ml), Vc2CBMTD (400 μg/mL), Vc4CBM (400 μg/mL) or PBS (control) and left for 24 hours. The cells were then subject to a BrdU assay and incubated for 2-6 hrs. An ELISA Assay was performed to measure the quantity of BrdU incorporated into the DNA. This assay is a standard assay to measure cell growth/proliferation. These graphs represent the percentage inhibition observed for (a) Sp2CBMTD (400 μg/mL), (b) Vc2CBM (400 μg/mL) and (c) Vc4CBM (400 μg/mL) vs their respective control (PBS treated) across the different cancer cell lines.

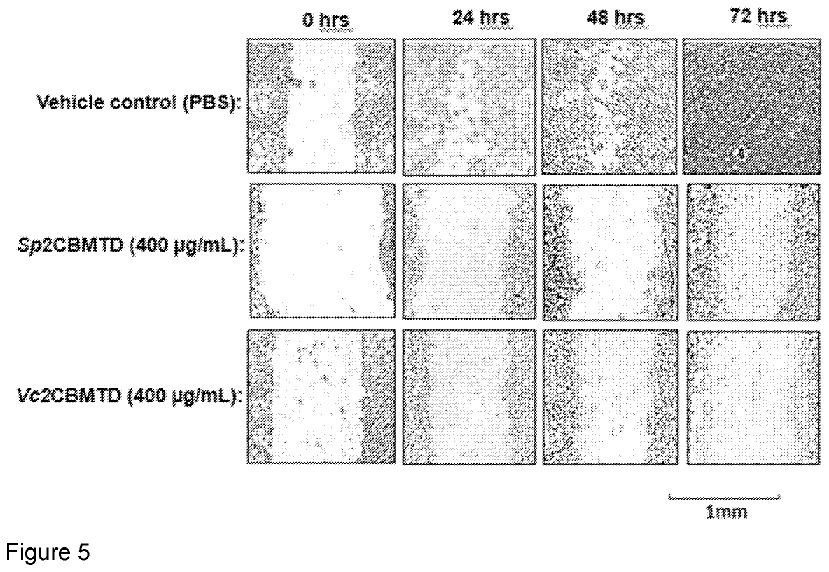

FIG. 5: Wound healing assay. Scratch assay data for human ovarian cancer cell line (SK-OV-3) after treatment with either PBS, Sp2CBMTD, or Vc2CBMTD (400 μg/mL). Images represent cell wound closure after 24 hrs, 48 hrs and 72 hrs. Conclusion: mCBM40 activity against metastatic ovarian cancer cell line, and consistent with data from other cancer cell lines.

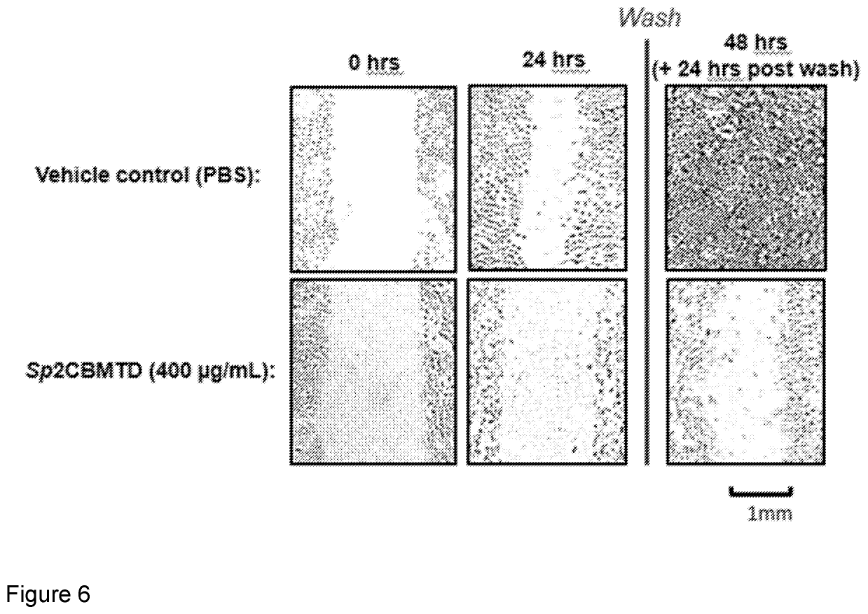

FIG. 6: Wound healing assay. Scratch assay data for human ovarian cancer cell line (SK-OV-3) after treatment with either PBS or Sp2CBMTD (400 μg/mL). After 24 hrs wounds were washed (3×5004 PBS) and fresh media (2% FBS, McCoy's 5A media) added. Wounds were then imaged 24 hrs after the treatment wash off. Images represent cell wound closure after 24 hrs, and 48 hrs (24 hrs after treatment wash-off). Conclusion: Sp2CBMTD has prolonged activity against metastatic ovarian cancer cell line SK-OV-3, even after treatment wash-off.

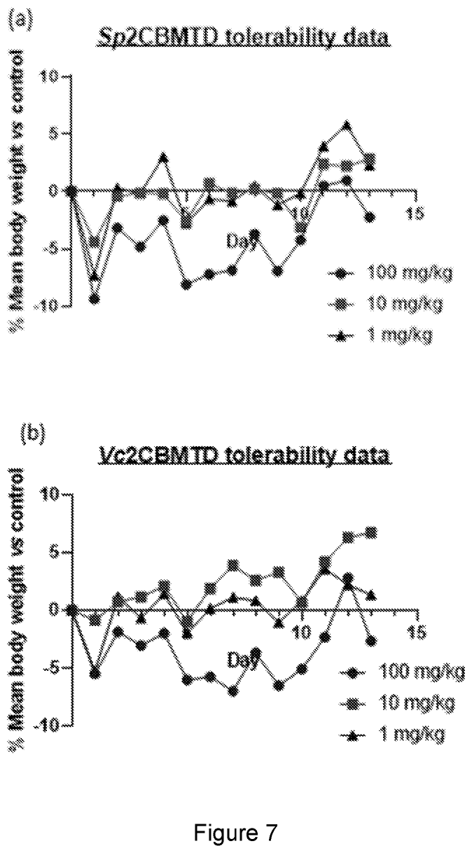

FIG. 7: In vivo tolerability data (a). NOG mouse tolerability data for Sp2CBMTD delivered once every four days (Q4D) through intraperitoneal delivery at 100, 10 and 1 mg/kg. (b). NOG mouse tolerability data for Vc2CBMTD delivered once every four days (Q4D) through intraperitoneal delivery at 100, 10, and 1 mg/kg. Data represented as body weight normalized to vehicle control group. Conclusion: Slight weight drop observed post dose, but weight recovery observed over time. Sp2CBMTD is well tolerated through intraperitoneal delivery at 10 mg/kg and 1 mg/kg. At 100 mg/kg Sp2CBMTD is tolerated.

### In Vivo Tolerability

TABLE 2
In vivo tolerability data. Animal Body Weight and General Tolerability
Data over the 14-Day Q4D Repeat Dosing Study using NOG mice. Conclusion:
Slight weight drop observed during study. No mortalities observed during study.

| | | | Day 13 Weight | | | | | |
|---|---|---|---|---|---|---|---|---|
| | | Dose | Mean ± SEM | | Weight Nadir | | Death | |
| Group | Treatment | (mg/kg) | (g) | % vD₀ | % vD₀₍ₘₐₓ₎ | Day | Total | Day |
| 1 | Sp2CBMTD | 1, IP | 17.50 ± 1.21 | −6.69 | −9.05 | 1 | 0 | — |
| | | Q4Dx4 | | | | | | |
| 2 | Sp2CBMTD | 10, IP | 19.09 ± 0.29 | −5.92 | −9.59 | 10 | 0 | — |
| | | Q4Dx4 | | | | | | |
| 3 | Sp2CBMTD | 100, IP | 17.30 ± 0.68 | −11.01 | −12.49 | 6 | 0 | — |
| | | Q4Dx4 | | | | | | |
| 4 | Vc2CBMTD | 1, IP | 17.92 ± 1.06 | −7.10 | −7.10 | 13 | 0 | — |
| | | Q4Dx4 | | | | | | |
| 5 | Vc2CBMTD | 10, IP | 19.02 ± 0.38 | −1.78 | −5.58 | 10 | 0 | — |
| | | Q4Dx4 | | | | | | |
| 6 | Vc2CBMTD | 100, IP | 18.519 ± 0.42 | −8.41 | −8.81 | 9 | 0 | — |
| | | Q4Dx4 | | | | | | |

TABLE 3
In vivo tolerability clinical score table. Clinical Score Data over the 14-Day
Q4D Repeat Dosing Study using NOG mice. Clinical observations scored as 1
(no adverse clinical observations), and 0 (clinical observations displayed).
Clinical observations noted = *Slightly hunched posture. ^{§}Slightly thin
appearance. Conclusion: Only minor clinical signs observed in a few animals.
mCBM40 determined to be tolerated in mice using IP repeated dosing.

| | | General Appearance Clinical Score | | | | | | | | | | | | | |
|---|---|---|---|---|---|---|---|---|---|---|---|---|---|---|---|
| | | Day | | | | | | | | | | | | | |
| Treatment | Animal # | 0 | 1 | 3 | 4 | 5 | 6 | 7 | 8 | 9 | 10 | 11 | 12 | 13 | 14 |
| Vehicle | 1.1 | 1 | 1 | | 1 | 1 | | | 1 | 1 | | | 1 | 1 | |
| | 1.2 | 1 | 1 | | 1 | 1 | | | 1 | 1 | | | 1 | 1 | |
| | 1.3 | 1 | 1 | | 1 | 1 | | | 1 | 1 | | | 1 | 1 | |
| Sp2CBMTD (1 | 2.1 | 1 | 1 | | 1 | 1 | | | 1 | 1 | | | 1 | 1 | |
| mg/kg, IP, Q4D) | 2.2 | 1 | 1 | | 1 | 0* | 0* | 0* | 0* | 0*^{§} | 0*^{§} | 0*^{§} | 0*^{§} | 0*^{§} | 0*^{§} |
| | 2.3 | 1 | 1 | | 1 | 1 | | | 1 | 1 | | | 1 | 1 | |
| Sp2CBMTD (10 | 3.1 | 1 | 1 | | 1 | 1 | | | 1 | 1 | | | 1 | 1 | |
| mg/kg, IP, Q4D | 3.2 | 1 | 1 | | 1 | 1 | | | 1 | 1 | | | 1 | 1 | |
| | 3.4 | 1 | 1 | | 1 | 1 | | | 1 | 1 | | | 1 | 1 | |
| Sp2CBMTD (100 | 4.1 | 1 | 1 | | 1 | 1 | | | 1 | 1 | | | 1 | 1 | |
| mg/kg, IP, Q4D) | 4.2 | 1 | 1 | | 1 | 1 | | | 1 | 1 | | | 1 | 1 | |
| | 4.3 | 1 | 1 | 0* | 0* | 0* | 0* | 0* | 0* | 0* | 0* | 0* | 0* | 0* | 0* |
| Vc2CBMTD (1 | 5.1 | 1 | 1 | | 1 | 1 | | | 1 | 1 | | | 1 | | |
| mg/kg, IP, Q4D) | 5.2 | 1 | 1 | | 1 | 1 | | | 1 | 1 | | | 1 | 1 | |
| | 5.3 | 1 | 1 | | 1 | 1 | | | 1 | 1 | | | 1 | 1 | |
| Vc2CBMTD (10 | 6.1 | 1 | 1 | | 1 | 1 | | | 1 | 1 | | | 1 | 1 | |
| mg/kg, IP, Q4D) | 6.2 | 1 | 1 | | 1 | 1 | | | 1 | 1 | | | 1 | 1 | |
| | 6.3 | 1 | 1 | | 1 | 1 | | | 1 | 1 | | | 1 | 1 | |
| Vc2CBMTD (100 | 7.1 | 1 | 1 | 0* | 0* | 0* | 0* | 0* | 0* | 0*^{§} | 0* | 0* | 0* | 0* | 0* |
| mg/kg, IP, Q4D) | 7.2 | 1 | 1 | | 1 | 1 | | | 1 | 1 | 0*^{§} | 0*^{§} | 0*^{§} | 0*^{§} | 0*^{§} |
| | 7.3 | 1 | 1 | | 1 | 1 | | | 1 | 1 | | | 1 | 1 | |

1.-23. (canceled)

24. A method of treating or preventing a cancer selected from the group consisting of:
(a) glioma;
(b) lung cancer;
(c) breast cancer;
(d) pancreatic cancer; and
(e) ovarian cancer,
the method comprising administering a sialic acid binding molecule to a subject in need thereof.

25. The method according to claim 24, wherein a sialic acid binding part of the sialic acid binding molecule comprises, consists essentially of, or consists of:
(i) a CBM; and/or
(ii) a CBM40; and/or
(iii) two, three, four or more CBMs; and/or
(iv) two, three or four more CBM40s.

26. The method according to claim 24, wherein the sialic acid binding molecule comprises the sialic acid binding domain of Vibrio cholerae NanH sialidase and/or the sialic acid binding domain of Streptococcus pneumoniae NanA sialidase.

27. The method according to claim 26, wherein the Vibrio cholerae NanH sialidase comprises the amino acid sequence of SEQ ID NO: 1 or 2.

28. The method according to claim 26, wherein the Streptococcus pneumoniae NanA sialidase comprises the amino acid sequence of SEQ ID NO: 3 or 4.

29. The method according to claim 26, wherein the sialic acid binding molecule comprises two or more Vibrio cholerae NanH sialidases and/or two or more Streptococcus pneumoniae NanA sialidases.

30. The method according to claim 26, wherein the sialic acid binding molecule comprises 2, 3, 4, 5, 6, 7, 8, 9 or 10 Vibrio cholerae NanH sialidases and/or Streptococcus pneumoniae NanA sialidases.

31. The method according to claim 24, wherein the sialic acid binding molecule is a sialic acid binding protein or a sialic acid binding portion thereof which comprises, consists essentially of, or consists of Vc2CBM; or Vc4CBM; or Sp2CBM; or Vc2CBMTD; or Vc4CBM or Sp2CBMTD.

32. An in vitro method of modulating the expression, function and/or activity of one or more gene(s), said method comprising contacting a cell or cells with a sialic acid bind molecule.

33. The method of claim 32, wherein the one or more gene(s) is/are selected from the group consisting of:
(a) gene(s) associated with or related to DNA damage repair;
(b) gene(s) associated with cell metabolism;
(c) gene(s) associated with or related to the cell cycle;
(d) gene(s) associated, related to or encoding transcriptional repressor(s);
(e) gene(s) associated with or related to apoptosis;
(f) gene(s) associated with or related to cell adhesion;
(g) genes associated with or related to cell motility;
(h) gene(s) associated with or related to microtubule regulation; and
(i) gene(s) associated with, related to or encoding cell growth factors.

34. The method of claim 32, wherein the one or more gene(s) is/are selected from the group consisting of:
(a) ERCC5
(b) DDB2
(c) DKC1
(d) ERCC3 and
(e) POLB

35. The method of claim 32, wherein the one or more gene(s) is/are selected from the group consisting of:
(a) SLC2A1;
(b) ARNT;
(c) ATP5A1;
(d) COX5A;
(e) G6PD;
(f) LDHA;
(g) UQCRFS1;
(h) GAPDH and
(i) HPRT1.

36. The method of claim 32, wherein the one or more gene(s) is/are selected from the group consisting of:
(a) AURKA;
(b) BMP1;
(c) CCND3;
(d) CDC20;
(e) E2F4;
(f) MCM2;
(g) MK167;
(h) PINX1;
(i) SKP2; and
(j) WEE1.

37. The method of claim 32, wherein the one or more gene(s) comprises or is SNAI3.

38. The method of claim 32, wherein the one or more gene(s) is/are selected from the group consisting of:
(a) ANOL3;
(b) CASP2; and/or
(c) CASP7.

39. The method of claim 32, wherein the one or more gene(s) comprises or is CDH2.

40. The method of claim 32, wherein the one or more gene(s) comprises or is TEP1.

41. The method of claim 32, wherein the one or more gene(s) comprises or is ACTB.

42. The method of claim 32, wherein the one or more gene(s) comprises or is VEGFC.

43. The method according to claim 24 wherein the sialic acid binding molecule is Vc2CBM and the cancer is selected from the group consisting of:
(a) pancreatic cancer; and
(b) breast cancer.

44. The method according to claim 24 wherein the sialic acid binding molecule is Vc4CBM and the cancer is selected from the group consisting of:
(a) breast cancer; and
(b) lung cancer.

45. The method according to claim 24 wherein the sialic acid binding molecule is Sp2CBM and the cancer is selected from the group consisting of:
(a) glioma
(b) breast cancer; and
(c) lung cancer.

46. The method according to claim 24 wherein the sialic acid binding molecule is Sp2CBMTD and/or Vc2CBMTD and the cancer is ovarian cancer.
